(19) United States
(12) Patent Application Publication
(10) Pub. No.: US 2005/0288230 A1
(43) Pub. Date: Dec. 29, 2005

(54) Non-steroidal anti-androgen therapy

(75) Inventor: Bo-Eric Persson, Copenhagen (DK)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 10/514,662

(22) PCT Filed: May 13, 2003
(86) PCT No.: PCT/GB03/02057

§ 371 Date: May 19, 2005

(30) Foreign Application Priority Data
May 17, 2002 (GB) 0211280.3

(51) Int. Cl. A61K 38/09

(52) U.S. Cl. 514/16

(57) ABSTRACT

The present invention relates to a method of enhancing bone mineral density (BMD) in a patient in need thereof by administration of an effective amount of a non-steroidal anti-androgen compound, preferably bicalutamide. Further aspects of the invention include methods of treating prostate cancer in patients at increased risk of bone fracture and the use of anti-androgen compounds, in particular bicalutamide, in the manufacture of a pharmaceutical product for such purposes.

This application is a national stage filing under 35 U.S.C. 371 of International Application No. PCT/GB03/02057, filed May 13, 2003, which claims priority from United Kingdom Application No. 0211280.3, filed May 17, 2002, the specification of which is incorporated by reference herein. International Application No. PCT/GB03/02057 was published under PCT Article 21(2) in English.

### SUMMARY OF THE INVENTION

The present invention arises from the observation that administration of the non-steroidal anti-androgen Casodex™ (bicalutamide) to patients surprisingly causes an increase in bone mineral density (BMD). Accordingly, the present invention relates to a method of enhancing BMD in a patient in need thereof by administration of an effective amount of a non-steroidal anti-androgen compound, preferably bicalutamide. Further aspects of the invention include methods of treating prostate cancer in patients at increased risk of bone fracture and the use of anti-androgen compounds, in particular of 4′-cyano-α′,α′,α′-trifluoro-3-(4-fluorophenylsulphonyl)-2-hydroxy-2-methylpropiono-m-toluidide, in the manufacture of a pharmaceutical product for such purposes.

### BACKGROUND TO THE INVENTION

One of the conventional treatments for patients suffering from prostate cancer is surgical (orchiectomy) or chemical castration, utilising for example a luteinising hormone releasing hormone (LHRH) agonist such as goserelin, buserelin, leuprorelin or triptorelin, to inhibit gonadotrophin secretion. Castration is however, associated with significant decreases in bone mineral density (BMD), placing patients at increased risk of osteoporotic fractures (Stoch et al., J. Clinical Endocrinology & Metabolism. 86(6):2787-2791, 2001; Kiralti et al. Urology. 57(1):127-132, 2001). For example, Kiralti et al. teach that mean baseline hip BMD is approximately 0.94 g/cm² which drops to about 0.8 g/cm² in patients on androgen deprivation therapy.

Bicalutamide, a non-steroidal anti-androgen, is the racemate of 4′-cyano-α′,α′,α′-trifluoro-3-(4-fluorophenylsulphonyl)-2-hydroxy-2-methylpropiono-m-toluidide. Bicalutamide is known by the AstraZeneca trade name CASODEX™. EP-100172 discloses 4′-cyano-α′,α′,α′-trifluoro-3-(4-fluorophenylsulphonyl)-2-hydroxy-2-methylpropiono-m-toluidide (named in EP-100172 as 4-cyano-3-trifluoromethyl-N-(3-p-fluorophenylsulphonyl-2-hydroxy-2-methylpropionyl)aniline) as the 8^{th} compound listed in the table in Example 6. The corresponding structure is shown in formula I:—

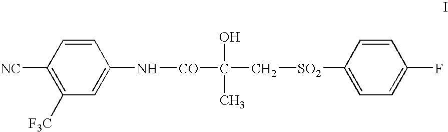

Bicalutamide can be used to combat prostate cancer, and the properties and usefulness of bicalutamide as an anti-androgen have been reviewed in B J A Furr et al., Urology, 1996, 47 (Suppl. 1A), 13-25, and G J C Kolvenbag et al., Urology, 1996, 47 (Suppl. 1A), 70-79.

Other examples of anti-androgens used in the treatment of prostate cancer are flutamide and nilutamide. The properties and usefulness of these anti-androgens have been reviewed, for example in the following documents which are incorporated herein by way of reference:—
flutamide R 0 Neri, J. Drug Develop., 1987, 1 (Suppl.), 5-9 and Urology, 1989, 34 (Suppl. 4), 19-21 and United Kingdom Patent Application No. 1360001;
nilutamide M G Harris et al., Drugs and Aging, 1993, 3, 9-25 and United Kingdom Patent Application No. 1518444.

It has been observed that the administration of bicalutamide in single agent therapy to humans causes an increase in the amount of testosterone circulating in the blood. Blackledge et al, (Urology, 1996, 47, Suppl. 1A), pp 44-47) disclose an approximate doubling of the basal level of total testosterone. It is believed that such an increase in the level of testosterone occurs when sufficient of the anti-androgen gains access to the CNS and blocks androgen receptors in the hypothalamus. The consequential lack of feedback of androgen causes additional release of LHRH by the hypothalamus which in turn causes release of luteinising hormone (LH) and follicle stimulating hormone (FSH) by the pituitary gland and consequently stimulation of production of testosterone in the testes. Aromatase enzyme in fat and other tissues converts some of the increased concentration of testosterone to oestradiol, which results in increased concentrations of oestrogen in the blood. Further discussion of this is provided by C Mahler et al, Clinical Pharmacokinetics, 1998, 34(5), pp 405-417.

The observation that Casodex monotherapy maintains BMD or does not cause a significant decrease in BMD has been reported previously. Iversen et al. (J Urology 164:1579-1582, 2000) reported that “the BMD of the proximal femur and total hip was similar to age matched general population, whereas castrated patients had clinically significantly reduced bone mineral density compared with age matched general population”. Abrahamsson (Eur. Urology, 39 (suppl. 1):22-28, 2001) reported that preliminary data from a locally advanced prostate cancer trial for Casodex 150 monotherapy and stated that the data suggested that bicalutamide maintains bone mineral density. As far as the inventors are aware however, this patent application is the first teaching that administration of a non-steroidal anti-androgen, such as bicalutamide, can effect a statistically significant increase in BMD, opening up new opportunities for the therapeutic use of non-steroidal anti-androgens.

Osteoporosis is a disease characterised by low bone mass and structural deterioration of bone tissue leading to bone fragility and an increased susceptibility to fractures of the hip, spine, ribs and wrist. Osteoporosis and low bone mass is a major health threat for an estimated 44 million Americans aged 50 years and over, 80% of whom are women. Ten million individuals are estimated to have the disease and 34 million more are estimated to have low bone density, placing them at increased risk for osteoporosis. In the USA, osteoporosis and low bone mass affected over 14 million men in 2002, of whom approximately 2 million had osteoporosis. One in two women and one in eight men aged 50 and over will have an osteoporosis-related fracture in their lifetime. The estimated national direct expenditures (hospitals and nursing homes) in the United States for osteoporotic and associated fractures was $17 billion in 2001.

While osteoporosis is often thought of as an older person's disease, it can strike at any age.

In addition, drug treatments for certain diseases have the unfortunate side effect of reducing BMD, thus increasing the risk of osteoporosis and bone fracture. Examples of such treatment are hormonal therapies, for example those involving administration of a luteinising hormone releasing hormone (LHRH) agonist or antagonist, used in the treatment of, inter alia, prostate cancer and gonadotrophin analogues used to treat endometriosis. Use of corticosteroids and anticonvulsants also increase the risk of developing osteoporosis.

There is considerable evidence that fractures can result from low bone mass because BMD accounts for 75-85% of the variance in the strength of bone. The accurate measurement of bone mass is therefore important in the diagnosis and monitoring of patients with osteoporosis. Dual-energy X-ray absorptiometry (DEXA) is widely used and can measure BMD in the hip and spine, both of which are important sites of osteoporotic fractures. Bone density measurements have been shown to be able to predict the risk of fractures related to moderate trauma, with age-adjusted relative risks ranging from 1.4 to 1.6 per one SD decrease in bone mass. Measurement of BMD is, therefore, a valuable method for identifying at-risk patients who may benefit from therapies which preserve or increase bone mass, and additionally measurement of BMD can be used, alone or in combination with biochemical markers, to monitor responses to treatment.

An individuals' BMD can be compared to two “norms”, “young normal” and “age matched”. Young normal, known as the T-score, compares actual BMD to optimal or peak density of a 30-year old healthy adult and determines the individual's fracture risk, which increases as BMD falls below young-normal levels. Age matched, known as the Z-score, compares actual BMD to what is expected in someone of the same age and body size. However, among older adults, low BMD is common, so comparison with age-matched norms can be misleading. Accordingly, comparison with T-score is usually conducted.

The difference between an individuals' BMD and that of a healthy young adult is referred to as a standard deviation (SD). As outlined in the World Health Organisation's diagnostic categories, individuals whose T-score is from −1 to −2.5 SD of the “norm” are considered to have osteopenia, and those with a score of and below −2.5 SD of the “norm” are considered to have osteoporosis. For most BMD tests, −1 SD approximates a 10% decrease in BMD.

Of relevance is the finding that, as disclosed in the Examples herein, 2 years treatment with an LHRH agonist results in an approximate 5% decrease in BMD in male patients. Therefore, a nominal patient that possesses a T-score of −0.5 SD before initiation of medical castration treatment might well after 2 years or more of treatment possess a T-score of <−1 SD, and thus be classified as osteopoenic with a much greater bone fracture risk.

There is therefore a need in the art for new medicaments to help treat osteoporosis and to help reduce the risk of developing osteopenia, osteoporosis and bone fractures. The inventors have surprisingly found that administration of the non-steroidal anti-androgen drug Casodex can result in an increase in BMD and thus a reduction in the individuals' bone-fracture risk.

### DETAILED DESCRIPTION OF THE INVENTION

According to a first aspect of the invention there is provided a method of enhancing bone mineral density (BMD) in a patient in need thereof comprising administration of an effective amount of a non-steroidal anti-androgen.

Diseases or conditions associated with a decreased BMD and increased disposition to bone fracture, include: osteoporosis, osteopenia, osteogenesis (including osteogenesis imperfecta), Paget's disease, hyperparathyroidism, hyperthyroidism, hypogonadism, hereditary bone demineralisation diseases, fibrous dysplasia and chronic inflammatory disorders (e.g. rheumatoid arthritis). In addition to these, certain therapeutic agents also cause a decrease in BMD. Examples include, patients on long-term glucocorticosteroids (e.g. for severe asthma, rheumatoid arthritis or chronic obstructive pulmonary disease), patients on gonadotrophin releasing hormone (GnRH) analogues to treat endometriosis, patients being treated with anticonvulsants and patients on long-term heparin treatment. Patients undergoing long-term kidney dialysis are also at risk of osteopenia/osteoporosis. Furthermore, lack of UV light, malabsorption/malnutrition, alcoholism and smoking can also decrease BMD. Accordingly, such patients may also benefit from the present invention.

Typical suitable non-steroidal anti-androgens include imidazolines, particularly 1-(3′-trifluoromethyl-4′-nitrophenyl)-4,4-dimethylimidazoline-2,5-dione (known as nilutamide) described in U.S. Pat. No. 4,097,578, or 4′-nitro-3-trifluoromethylisobutyranilide (known as flutamide) described in U.S. Pat. No. 4,329,364, hydroxyflutamide described in U.S. Pat. No. 3,875,229 and prodrug forms of hydroxyflutamide as well as N-(phenylalkanoyl)aniline derivatives disclosed in U.S. Pat. No. 4,386,080; the 3,4-disubstituted-branched-chain acylanilides disclosed in U.S. Pat. No. 4,239,776 and U.S. Pat. No. 4,636,505 (which includes bicalutamide).

Steroidal anti-androgens such as 6-chloro-1,2-dihydro-17-(acetyl)-3′H-cyclopropa[1,2]-pregna-1,4,6-triene-3,20-dione (known as cyproterone acetate) are excluded from the present invention. Steroidal molecules, like cyproterone acetate and chlormadinone acetate, reduce LH and testosterone and cause castration-like effects, and so, especially with long-term treatment, will likely accelerate bone degradation. Accordingly, it is not believed that steroidal anti-androgen molecules will work according to the present invention.

In one embodiment, the non-steroidal anti-androgen for use in each aspect of the invention is selected from the group consisting of: bicalutamide, nilutamide and flutamide.

In a further embodiment the non-steroidal anti-androgen for use according to each aspect of the invention is a pure non-steroidal anti-androgen.

By the term “pure non-steroidal anti-androgen” is meant a non-steroidal anti-androgen which is devoid of any androgenic, estrogenic, progestational, anti progestational, antigonadotrophic or adrenocortical activity.

By the term “effective amount” is meant the dosage which is sufficient to bring about the desired effect (e.g. increase in BMD, reduce bone fracture risk, treat prostate cancer etc.), such amounts can be determined via clinical trials by the person skilled in the art without undue experimentation or inventive effort.

The term “treating prostate cancer” as used herein means treating, alleviating or palliating such condition, suppressing the growth of cancerous tissue and thus providing an increase in survival time.

According to a further aspect of the invention there is provided a method of treating a patient suffering from or likely to develop osteopenia or osteoporosis, comprising administration to said patient of an effective amount of a non-steroidal anti-androgen.

The terms “osteoporosis” and “osteopenia” as used herein, refer to the condition characterised by reduced bone mass and disruption of bone architecture, resulting in increased bone fragility and increased fracture risk. Both may be recognised by bone mineral density measurements. According to this characterisation, osteoporosis is defined as a bone mineral density in the spine and/or proximal femur 2.5 or more standard deviations below young (30 years) adult mean peak bone mass (T-score). Osteopenia is characterised as being between one (1) and 2.5 SD below young (30 years) adult mean peak bone mass (T-score).

By the term “a person likely to develop.” as used herein means a person who currently does not suffer the condition/disorder (e.g. osteopenia or osteoporosis) but who can be classified via secondary surrogate markers (BMD score or biochemical markers) as likely to develop the condition/disorder.

According to a further aspect of the invention there is provided a method of reducing an individuals' bone fracture risk comprising administration of an effective amount of a non-steroidal anti-androgen.

It is well established that treatment of prostate cancer by surgical or chemical/medical castration causes a reduction in BMD and thus increases the bone fracture risk of the patient. The present Example shows that patients receiving medical castration experienced a progressive 4-5% loss of BMD over a 96-week period. The finding that Casodex monotherapy treatment is, not only as effective as castration in treating prostate cancer, but also, offers the additional benefit of increasing BMD, potentially opens up new treatment regimes. For example, patients currently or previously on LHRH treatments that have suffered significant BMD decrease (and thus increased fracture risk) can be switched to non-steroidal pure anti-androgen (e.g. Casodex) treatment. Alternatively, alternating treatment regimes of medical castration and anti-androgen therapy can be envisaged. The anti-androgen regime allowing the BMD to recover. Such regimes may, for example, comprise monthly alternating regimes of treatment wherein the patient is treated with an LHRH agonist for one month and then with a non-steroidal anti-androgen for a further month. Of course, alternating regimes of different duration can also be envisaged, such as for example five weeks with the first treatment and three months with the second. Longer regime intervals can also be envisaged. For example the first treatment interval may be 6 months or longer, then stopped, and the second treatment started. A treatment vacation (where the patient is not subjected to any treatment, for example to recover from any side effects) may also be incorporated into the alternating regime. The alternating regime can be devised by the physician.

According to a further aspect of the invention there is provided a method of enhancing bone mineral density in a prostate cancer patient previously treated with an LHRH agonist comprising administration of an effective amount of a non-steroidal anti-androgen.

According to a further aspect of the invention there is provided a method of reducing the bone fracture risk in a prostate cancer patient comprising subjecting the patient to a treatment regime that involves alternating regimes of LHRH agonist treatment and non-steroidal anti-androgen (esp. bicalutamide) monotherapy treatment.

Because chemical and medical castration causes a progressive decrease in BMD, those patients that already have BMD T-scores below the norm are more susceptible to developing osteoporosis and have greater bone fracture risk. Accordingly, it would be advantageous to determine the T-score of the prostate cancer patient and elect to adopt an anti-androgen treatment regime for those patients that possess a negative T-score. In alternate embodiments an anti-androgen regime would be adopted for those patients with a T-score of and below, −0.25 SD, −0.35 SD, −0.4 SD, −0.45 SD, −0.5 SD, −0.75 SD, −1.0 SD and −1.5 SD. This is because castration therapy for such patients would decrease their T-score even further with the potential to be classified as osteopenic or osteoporotic.

Thus, according to a further aspect of the invention there is provided a method of treating prostate cancer in a patient that exhibits a BMD T-score of less than or equal to −0.25 SD, comprising administering to said patient an effective amount of a non-steroidal anti-androgen.

According to a further aspect of the invention there is provided a method of treating prostate cancer in a patient comprising measuring the BMD score of a patient suffering from or being treated for prostate cancer, selecting a patient that exhibits a BMD T-score of less than or equal to −0.25 SD and administering to said patient an effective amount of a non-steroidal anti-androgen.

It will be appreciated that these aspect of the invention (treatment of prostate cancer patients with sub-normal BMD) is equally applicable to other diseases that are treated using non-steroidal anti-androgens like bicalutamide. Such diseases include non-malignant disease of the prostate gland (eg, benign prostatic hyperplasia or hypertrophy), hirsutism and acne.

There are several ways to measure bone mineral density; all are painless, noninvasive and safe and are becoming more readily available. The tests measure bone density in the spine, hip and/or wrist, the most common sites of fractures due to osteoporosis. Recently, bone density tests have been approved by the FDA that measure bone density in the middle finger and the heel or shinbone.

The information from a bone density test can be used to determine the patient's fracture risk and diagnose osteopenia or osteoporosis. In general, the lower the BMD value, the higher the risk for fracture.

There are several different machines that measure bone density. Central machines measure density in the hip, spine and total body. Peripheral machines measure density in the finger, wrist, kneecap, shin-bone and heel.

DEXA (Dual Energy X-ray Absorptiometry) measures the spine, hip or total body; pDEXA (Peripheral Dual Energy X-ray Absorptiometry) measures the wrist, heel or finger; SEXA (single Energy X-ray Absorptiometry) measures the wrist or heel; QUS (Quantitative Ultrasound) uses sound waves to measure density at the heel, shin bone and kneecap; QCT (Quantitative Computed Tomography) most commonly used to measure the spine, but can be used at other sites; pQCT (Peripheral Quantitative Computed Tomography) measures the wrist; RA (Radiographic Absorptiometry) uses an X-ray of the hand and a small metal wedge to calculate bone density; DPA (Dual Photon Absorptiometry) measures the spine, hip or total body (used infrequently); and, SPA (Single Photon Absorptiometry) measures the wrist (used infrequently).

Of course it will be appreciated that BMD determination is not the only means of determining whether or not a patient has a high or low fracture risk. Biochemical tests are available to measure the levels of biochemical markers (i.e. bone proteins such as the pyridinoline and deoxypyridinoline crosslinks of collagen), whose levels in the blood are indicative of bone degradation.

Bone is comprised of extracellular matrix proteins which are physiologically mineralised with calcium salts. Approximately 85-90% of the total protein within bone consists of type I collagen. The remaining 10-15% of protein is comprised of noncollagenous proteins including proteoglycans, osteocalcin, osteonectin, osteopontin and bone sialoprotein as well as enzymes such as alkaline phosphatase.

Unlike most connective tissues, the bone matrix is constantly regenerated throughout life by a process of resorption and formation. The cycle of resorption and formation releases degradation products from a number of bone proteins. These degradation products can be used to monitor bone turnover and to determine whether, as in osteoporosis, there is an imbalance in which resorption exceeds formation, leading to a loss of bone mass.

Bettica and Moro (JIFCC. 7(1):16-22, 1995) describe the various biochemical markers of the bone that may be used to study bone metabolism and characterize bone fracture risk of a patient.

Aubrey Stoch et al. (infra) teach that urinary NTx and serum BSAP were both significantly higher in men receiving GnRH-agonist therapy, and thus possessing BMD deterioration, than those without treatment. Furthermore, they report that bone resorption and formation markers remain remarkably stable in healthy men beyond the third decade of life (citing: Fatayerji and Eastell. Age-related changes in bone turnover in men. J. Bone mineral Res. 14:1203-1210, 1999; and, Orwoll et al. J. Clin. Endocrin. Metab. 83:3930-3935, 1998). Garnero et al. (J. Bone Mineral Res. 11:337-349, 1996) in a cross-sectional study of women in France, found that with increasing time after menopause, the telopeptide markers of resorption, NTx and CTx, correlated negatively and increasingly strongly with BMD of the spine, hip, forearm, and total body. It is, therefore, believed that biochemical markers of bone turnover have a place in the prediction of risk for osteoporosis and in monitoring therapy in patients.

Serum and urinary markers of bone resorption such as the pyridinoline and deoxypyridinoline crosslinks of collagen can be measured by HPLC or by immunological techniques. These markers have been shown to correlate with bone turnover assessed by calcium kinetics and quantitative histological analysis, and their levels are markedly reduced by anti-resorptive drugs such as bisphosphonates.

Immunoassays for serum osteocalcin and bone-specific alkaline phosphatase can be used to measure bone formation.

The availability of biochemical markers permits the identification of patients who have high bone turnover and who may be at increased risk of osteoporosis and would therefore benefit from further investigations such as measurement of bone mineral density. Such biochemical markers can therefore be used to identify patients with reduced BMD that would benefit from the present invention. Additionally, biochemical markers permit the effects of therapies that may affect bone turnover to be monitored.

Accordingly, the methods of the invention requiring BMD determination can equally be employed using biochemical tests to determine the bone fracture risk.

Examples of suitable biochemical markers capable of determining whether or not a subject is or has recently suffered a breakdown in bone tissue include, urinary pyridinoline, urinary deoxypyridinone, BSAP and the telopeptide markers NTx and CTx.

Thus, according to a further aspect of the invention there is provided a method of treating prostate cancer in a patient at increased risk of bone fracture than the norm, comprising measuring the bone fracture risk of the patient suffering from or being treated for prostate cancer, selecting a patient that is at increased risk of bone fracture than the norm and administering to said patient an effective amount of a non-steroidal anti-androgen.

The term “the norm”, used according to this aspect of the invention, refers to the age-matched risk (Z-score).

According to a further aspect of the invention there is provided a method of treating osteoporosis in a patient in need of such treatment comprising administration of an effective amount of a non-steroidal anti-androgen, optionally in combination with an oestrogen.

According to a further aspect of each distinct aspect of the invention, the non-steroidal anti-androgen, particularly bicalutamide, is co-administered with tamoxifen.

Administration of tamoxifen is known to result in an increase in testosterone and oestradiol. In this respect, reference is made to F H Comhaire et al, Human Reproduction, 10(7):1740-1744, 1995, where tamoxifen intake in adult men was reported to increase testosterone and LH. Such additional increased levels could give added benefit in terms of BMD.

According to a further aspect of the invention there is provided a method of selecting whether or not a prostate cancer patient should be treated via castration or anti-androgen therapy comprising measuring the BMD score of the patient and selecting those patients with a T-score of less than or equal to −0.25 SD for treatment with an effective amount of a non-steroidal anti-androgen.

According to another aspect, the invention relates to the use of a non-steroidal anti-androgen compound in the manufacture of a medicament for treating disease states associated with sub-normal BMD values in warm-blooded animals, particularly humans.

According to a further aspect of the invention there is provided the use of a non-steroidal anti-androgen compound in the preparation of a medicament for increasing the BMD in a patient in need thereof.

According to a further aspect of the invention there is provided the use of a non-steroidal anti-androgen compound in the preparation of a medicament for treating a patient suffering from osteopenia or osteoporosis.

According to a further aspect of the invention there is provided a non-steroidal anti-androgen compound for use in the treatment of osteopenia or osteoporosis.

According to another aspect, the invention provides a non-steroidal anti-androgen compound for use in the treatment of disease states associated with sub-normal BMD values in warm-blooded animals, particularly humans.

According to a further aspect, the invention provides a non-steroidal anti-androgen compound for use in increasing the BMD in a patient in need thereof.

It will be appreciated that reference herein to non-steroidal anti-androgen compounds includes pharmaceutically-acceptable salts thereof, solvates, acids, esters, prodrugs and the like, and formulations comprising an agent with anti-androgenic activity. Suitable salts are, for example acid addition salts, such as hydrochloride, hydrobromide, phosphate, acetate, fumarate, maleate, tartarate, citrate, oxalate, methanesulphonate orp-toluenesulphonate, or alkali metal salts such as sodium or potassium salts.

The patient is preferably a human, e.g. an adult male, but the treatment of other mammals is also contemplated.

Bicalutamide, 4′-cyano-α′,α′,α′-trifluoro-3-(4-fluorophenylsulphonyl)-2-hydroxy-2-methylpropiono-m-toluidide, can exist in distinct R- and S-enantiomeric forms. The R-enantiomer is the (−) isomer and is the pharmacologically active compound in vivo. For further details of the enantiomers, reference is made to Tucker and Chesterton, J. Med. Chem. 31, pp 885-887 (1988).

The chemical synthesis of racemic 4′-cyano-α′,α′,α′-trifluoro-3-(4-fluorophenylsulphonyl)-2-hydroxy-2-methylpropiono-m-toluidide is described in U.S. Pat. No. 4,636,505, and this disclosure is incorporated herein by reference. The R-enantiomer may be obtained by chromatographic separation of diastereomeric esters of chiral acids. For example, the R-enantiomer may be prepared by chromatographic separation using chiral chromatography. Another method involves resolution of the carboxylic acid precursor, 3-(4-fluorophenyl)-2-hydroxy-2-methylpropanoic acid, by fractional crystallisation of diastereomeric salts with chiral amines. The Tucker and Chesterton reference cited above discloses the chromatographic separation of the R- and S-enantiomers from racemic 4′-cyano-α′,α′,α′-trifluoro-3-(4-fluorophenylsulphonyl)-2-hydroxy-2-methylpropiono-m-toluidide. The method involves the chromatographic separation of R-camphanoyl esters of the racemate and their hydrolysis and oxidation to the R- and S-enantiomers. This disclosure is incorporated herein by reference specifically to provide an illustration of a method of obtaining the bicalutamide enantiomers for use in the present invention. Other methods will, however, be evident to the skilled addressee using routine techniques for the preparation of enantiomers.

The present invention extends to the use of racemate and optically active enantiomeric forms of non-steroidal anti-androgens that possess chiral centres.

As noted above, according to a preferred embodiment of each aspect of the invention the non-steroidal anti-androgen is bicalutamide. Bicalutamide is currently administered as the racemate. However, it has been established that the R-enantiomer contains substantially all of the anti-androgenic activity. Accordingly, in one embodiment of the compound, formulation or dose, ≧50%, ≧60%, ≧65%, ≧70%, ≧80%, ≧85%, ≧90%, ≧95%, ≧98% or ≧99% or thereabout of the 4′-cyano-α′,α′,α′-trifluoro-3-(4-fluorophenylsulphonyl)-2-hydroxy-2-methylpropiono-m-toluidide (bicalutamide) is provided in the form of the R-enantiomer. In a preferred embodiment, 100% or substantially 100% of the 4′-cyano-α′,α′,α′-trifluoro-3-(4-fluorophenylsulphonyl)-2-hydroxy-2-methylpropiono-m-toluidide is provided in the form of the R-enantiomer. By “substantially 100%” we mean that the 4′-cyano-α′,α′,α′-trifluoro-3-(4-fluorophenylsulphonyl)-2-hydroxy-2-methylpropiono-m-toluidide is provided as the pure R-enantiomer, or there is a trace (<1%) of the S-enantiomer present.

The person skilled in the art of pharmaceutical formulation is capable of designing a suitable formulation for administering the non-steroidal anti-androgen compound. The compounds or compositions for use according to the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing). Skin patches are also contemplated. Formulation in general is described in Chapter 25.2 of Comprehensive Medicinal Chemistry, Volume 5, Editor Hansch et al, Pergamon Press 1990.

A composition for use in the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or acetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

It is possible that the therapeutic potential (reduced inter-patient variability, enhanced bioavailability etc.) of the non-steroidal anti-androgen drug, particularly bicalutamide, can be enhanced by formulating the drug as a solid dispersion formulation. Thus, in one embodiment the non-steroidal anti-androgen drug is prepared as a formulation comprising the drug in solid dispersion with an enteric polymer having a pKₐ from 3 to 6, or PVP. Such formulations are disclosed in WO 02/067893, WO 02/080902 and PCT/GB02/04621, incorporated herein by reference.

In one embodiment, the enteric polymer is selected from hydroxypropyl methylcellulose acetate succinate (HPMCAS), hydroxpropyl methylcellulose acetate pthalate, hydroxypropyl methylcellulose acetate, hydroxypropyl methylcellulose succinate, a methacrylic acid copolymer, polyvinyl acetate phthalate (PVAP), cellulose acetate phthalate (CAP), methylcellulose acetate phthalate, ethyl cellulose acetate phthalate, hydroxypropyl cellulose acetate phthalate, hydroxypropyl methylcellulose phthalate (HPMCP), cellulose proprionate pthalate, hydroxypropyl cellulose butyrate pthalate, hydroxypropyl cellulose acetate pthalate succinate, hydroxypropyl methylcellulose trimellitate, cellulose acetate trimellitate (CAT), methylcellulose acetate trimellitate, ethyl cellulose acetate trimellitate, hydroxypropyl cellulose acetate trimellitate, hydroxypropyl methylcellulose acetate trimellitate, hydroxypropyl cellulose acetate trimellitate succinate, cellulose proprionate trimellitate, cellulose butyrate trimellitate, cellulose acetate terepthalate and cellulose actetate isopthalate.

The use of the term “hydroxypropyl methylcellulose phthalate polymer”, or HPMCP, is known to the skilled reader for classifying a group of polymers which share the same basic structural features and include such polymers as: hypromellose phthalate; methylhydroxypropylcellulosi pthalas; cellulose, hydrogen 1,2-benzenedicarboxylate, 2-hydroxypropyl methyl; as well as commercially available polymers HP-55™, HP-55S™ and HP-50™ (available from Shin-Etsu Chemical Industry Co., Ltd., Japan or appointed distributors).

Preferably the hydroxypropylmethylcellulose phthalate polymer has a molecular weight (Mw) from 20 kDa to 200 kDa, eg from 80 kDa to 130 kDa. In one embodiment, the Mw is less than 150 kDa, or less than 100 kDa. HP-50, HP-55 and HP-55S are polymers known in the literature and widely used as an enteric coating for oral formulations. HP-55 has a Mw 84 kDa. HP-55S has a Mw of 132 kDa. HP-50 has a Mw 78 kDa. HP-50 is soluble at pH≧5, whereas HP-55 and HP-55S are soluble at pH≧5.5. In one embodiment, the non-steroidal anti-androgen is in a solid dispersion with at least one polymer selected from HP-50, HP-55 and HP-55S. Thus, it is contemplated that a mixture of two or more of these HPMCP polymers can be used.

In a preferred embodiment, the non-steroidal anti-androgen is R-enantiomeric bicalutamide in solid dispersion with HP-55S polymer.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

Compositions for administration by insufflation may be in the form of a finely divided powder containing particles of average diameter of, for example, 30 μm or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 10 mg to 1 g of active agent compounded with an appropriate and convenient amount of excipients which may vary from about 5 to about 98 percent by weight of the total composition. Dosage unit forms will generally contain about 10 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

In using a non-steroidal anti-androgen compound according to the various aspects of the invention it will generally be administered so that a daily dose in the range, for example, 0.1 mg to 50 mg per kg body weight is received, given if required in divided doses. In general lower doses will be administered when a parenteral route is employed. Thus, for example, for intravenous administration, a dose in the range, for example, 0.25 mg to 10 mg per kg body weight will generally be used. Similarly, for administration by inhalation, a dose in the range, for example, 0.15 mg to 25 mg per kg body weight will be used. Oral administration is however preferred, particularly in tablet form. Typically, unit dosage forms will contain about 5 mg to 700 mg of an anti-androgen such as bicalutamide.

One aspect of the invention provides a daily pharmaceutical dose of bicalutamide mucosally administrable to a patient wherein the dose comprises 25 to 1000 mg of bicalutamide. Preferably, the dose comprises an upper limit of 750, 600, 500, 450, 400, 300, 200, 150, 125, 100, 75 or 50 mg of bicalutamide. In one example, the dose comprises 150 mg of bicalutamide.

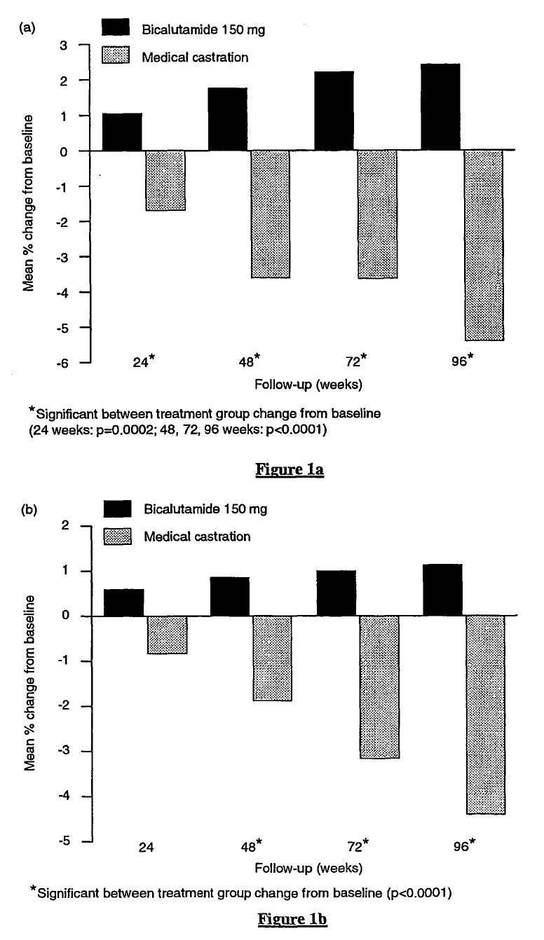

The invention will be further described by way of the following non-limiting examples and accompanying FIG. 1 which shows the percentage change from baseline in (a) lumbar spine-BMD and (b) hip-BMD at Weeks 24, 48, 72 and 96.

### EXAMPLES

Study Design

Inclusion criteria for a prospective, multicentre, randomized, open-label, parallel-group study, included: disease stage T1-T4, Nx, and M0 for which immediate hormonal ablation was indicated; and Eastern Cooperative Oncology Group (ECOG) performance status 0 or 1.

Patients were randomized in a 1:1 ratio to receive either bicalutamide 150 mg daily or medical castration with a luteinizing hormone-releasing hormone (LHRH) agonist for 2 years.

Assessments

Primary efficacy endpoints were percentage change from baseline in BMD measures (lumbar spine and hip) at 96 weeks.

An analysis of covariance (ANCOVA) model was used to analyse the primary efficacy endpoints. Analysis was performed for patients for whom data were available at baseline and at Week 96. Subsidiary analyses were performed, carrying forward the last patient assessment at time points where data were missing. A p value of ≦0.017 was considered statistically significant.

Secondary endpoints included: percentage change in lumbar spine-BMD and hip-BMD from baseline to 24, 48 and 72 weeks.

Secondary efficacy endpoints were also analyzed using an ANCOVA model. BMD were assessed via dual energy X-ray absorptiometry (DEXA).

Results

Patient Demography

103 patients were recruited to the study and randomized 1:1 to receive either bicalutamide (R/S racemate) 150 mg (n=51) or medical castration (n=52).
The treatment groups were well balanced at baseline (Table 1).
The median testosterone level at baseline was 414.7 ng/dL in the bicalutamide 150 mg group (n=48) and 477.9 ng/dL in the medical castration group (n=52).
Median baseline values for albumin, total calcium, 25-hydroxy vitamin D, and thyroid-stimulating hormone were similar in the two treatment groups.
   Bone Mineral Density

At Week 96, both lumbar spine- and hip-BMD were elevated relative to baseline in the bicalutamide 150 mg group (+2.42% and +1.13%, respectively), compared with BMD loss relative to baseline in the medical castration group (−5.40% and −4.39%, respectively). (Table 2; FIGS. 1a and 1b).

The difference between treatment groups was statistically significant for both measures at Week 96 (p<0.0001) [Table 2; FIGS. 1a and 1b].

At all other assessments (Weeks 24, 48 and 72), there was a statistically significant difference between treatments in percentage change from baseline in lumbar spine- and hip-BMD in favour of bicalutamide 150 mg, with the exception of hip-BMD at Week 24 (p=0.02) [FIGS. 1a and 1b].

Compared to pre-treatment baseline levels, lumbar spine BMD was progressively and significantly increased (p<0.05) on Casodex 150 mg at each of the time points assessed: 24 weeks: N=40, 1.05% increase, 48 weeks: N=35, 1.76% increase, 72 weeks: N=28, 2.22% increase, 96 weeks: N=24, 2.42% increase.

The hip values also show a consistent pattern with an increase in BMD over the time observed.

### CONCLUSIONS

The results, therefore, support our surprising finding that the administration of bicalutamide causes a statistically significant increase in BMD compared to baseline.

BMD was elevated in patients receiving bicalutamide 150 mg while those receiving medical castration experienced a progressive 4-5% loss during the 2 years' observation; the difference between treatments was highly significant.

TABLE 1
Patient demography

| | Bicalutamide 150 mg | Medical castration |
|---|---|---|
| | (n = 51) | (n = 52) |
| Mean age (range), years | 74.6 (53-87) | 75.2 (61-90) |
| Mean weight (range), kg | 80.0 (54-113) | 83.9 (55-116) |
| Race, % | | |
| White | 88.2 | 96.2 |
| Black | 9.8 | 3.9 |
| Hispanic | 2.0 | 0 |
| Disease stage, % | | |
| T1 | 27.5 | 19.2 |
| T2 | 39.2 | 51.9 |
| T3 | 31.4 | 26.9 |
| T4 | 2.0 | 1.9 |
| Nodal status, % | | |
| N0 | 54.9 | 67.3 |
| nx | 41.2 | 28.9 |
| N+ | 3.9 | 3.9 |

TABLE 2
Percentage change from baseline (standard error)
in lumbar spine-BMD, hip-BMD at Week 96

| | Bicalutamide | Medical | p value |
|---|---|---|---|
| | 150 mg | castration | (95% CI)* |
| Lumbar spine-BMD | 2.42 (0.88) | −5.40 (0.74) | <0.0001 |
| | [n = 24] | [n = 36] | (−10.15, −5.50) |
| Hip-BMD | 1.13 (0.60) | −4.39 (0.83) | <0.0001 |
| | [n = 24] | [n = 36] | (−7.79, −3.26) |

*95% CI for the mean difference for medical castration-bicalutamide 150 mg

1. A method of enhancing bone mineral density (BMD) in a patient in need thereof comprising administration to said patient of an effective amount of a non-steroidal anti-androgen.

2. A method of enhancing bone mineral density in a prostate cancer patient previously treated with luteinising hormone releasing hormone (LHRH) agonist comprising, administration of an effective amount of a non-steroidal anti-androgen.

3. A method of reducing the bone fracture risk in a prostate cancer patient comprising subjecting the patient to a treatment regime that involves alternating regimes of LHRH agonist treatment and non-steroidal anti-androgen monotherapy treatment.

4. A method of treating prostate cancer in a patient comprising selecting a patient that exhibits a BMD T-score of less than or equal to −0.25 SD and administering to said patient an effective amount of a non-steroidal anti-androgen.

5. A method of treating prostate cancer in a patient at increased risk of bone fracture than the T-score norm, comprising measuring the bone fracture risk of the patient suffering from or being treated for prostate cancer, selecting a patient that is at increased risk of bone fracture than the T-score norm and administering to said patient an effective amount of a non-steroidal anti-androgen.

6. The method according to claim 5, wherein the bone fracture risk is assessed by biochemical test or by measuring BMD of the patient.

7. The method according to claim 6, wherein the biochemical test involves measurement of amount of one or more of the following markers: urinary pyridinoline, urinary deoxypyridinone, BSAP and the telopeptide markers NTx and CTx.

8. (canceled)

9. A method of treating osteopenia or osteoporosis in a patient in need thereof comprising administration to said patient of an effective amount of a non-steroidal anti-androgen.

10. A method for treating a disease states associated with a sub-normal BMD values in a warm-blooded animals comprising administration to said animal of an effective amount of a non-steroidal anti-androgen.

11. The method or use according to any one of claims 1-3, wherein the non-steroidal anti-androgen is a pure non-steroidal anti-androgen.

12. The method or use according to claim 11, wherein the non-steroidal anti-androgen is selected from the group consisting of: bicalutamide, nilutamide and flutamide.

13. The method or use according to claim 12, wherein the non-steroidal anti-androgen is bicalutamide substantially in the R-enantiomeric form.

14. The method according to claim 1, wherein the patient is suffering from prostate cancer.

15. The method according to claim 1, wherein the patient has previously been treated with chemical or surgical castration.
